(19) United States
(12) Patent Application Publication
(10) Pub. No.: US 2006/0122144 A1
(43) Pub. Date: Jun. 8, 2006

(54) Compounds resistant to metabolic deactivation and methods of use

(76) Inventors: Robert Ronald Kane, Waco, TX (US); Hui-Min Chang, Edmonton (CA)

Correspondence Address: LAHIVE & COCKFIELD, 28 STATE STREET, BOSTON, MA, 02109, US

(21) Appl. No.: 11/145,550

(22) Filed: Jun. 2, 2005

Related U.S. Application Data
(63) Continuation of application No. PCT/US03/38638, filed on Dec. 3, 2003.
(60) Provisional application No. 60/430,397, filed on Dec. 3, 2002.

(51) Int. Cl. A61K 31/7076; C07H 19/16

(52) U.S. Cl. 514/46; 536/27.3

(57) ABSTRACT

Therapeutic compounds having increased resistance to deamination and inactivation by metabolic enzymes are provided. The compounds include nucleotide analogs and nucleotide analogs, derivatized with aminal and/or thioaminal groups to prevent deamination of free amine. The compounds can be used in a variety of treatments, including treatment of neoplastic disorders, infections from fungal or fungal-like organisms, and infections from parasites.

### RELATED APPLICATIONS

This patent application is a continuation of International Patent Application No. PCT/US2003/038638, filed Dec. 3, 2003, which claims the priority of U.S. Provisional Application No. 60/430,397, filed Dec. 3, 2002. The entire contents of the above-referenced patent applications are hereby incorporated herein by reference.

### BACKGROUND OF THE INVENTION

The clinical effectiveness of therapeutic compounds is dependent not only on the activity of the compound itself, but also on the bioavailability of the compound or its propensity to achieve therapeutically relevant concentrations in the bloodstream of the patient before metabolism eliminates the compound from the body. The problem of poor bioavailability is a significant limitation to the clinical development of a number of potentially useful therapeutic compounds. In these cases, large doses or continuous administration of drug is necessary to ensure that effective concentrations will be attained in the patient. These strategies are often associated with significant adverse effects.

One class of therapeutic compounds especially prone to inactivation by metabolic enzymes, are the nucleotides, nucleosides and analogs thereof. These analogs are important treatment options for cancer and viral infections due to their ability to incorporate into DNA or RNA during replication or transcription and inhibit further DNA or RNA synthesis. The effectiveness of certain of these compounds, especially those incorporating cytosine or adenosine, is therefore severely limited due to rapid deactivation by deaminases, which remove the free amines which are necessary for base pairing and proper integration into DNA. It is thus highly desirable to provide nucleoside analogs in a form which will persist for a longer period of time in a patient's body without degrading.

Cordycepin (3′-deoxyadenosine) is one example of an adenosine nucleoside analog that has a number of biological effects, including antiviral activity (Richardson et al. (1975) Int. J. Cancer 15:451-6), anti-cancer or anti-tumorigenic activity (Jagger et al. (1961) Cancer Res. 21:216-20), anti-inflammatory activity (Zhou et al. (2002) European Journal of Pharmacology 453:309-17), anti-fungal activity (Sugar et al. (1998) Antimicrobial Agents and Chemotherapy 42(6):1424-7; U.S. Pat. No. 5,679,648) and anti-parasitic activity (Trigg et al. (1971) Trans. R. Soc. Trop. Med. Hyg. 65:514-20; U.S. Pat. No. 5,663,155]. For one mechanism of action Cordycepin must be sequentially phosphorylated intracellularly by adenosine kinase and adenylate kinase to form 3′-deoxyadenosine triphosphate (3′deoxyATP). 3′-deoxyATP exerts many of its anticancer effects due to incorporation into RNA in lieu of ATP, thereby acting as a chain terminator during transcription in rapidly dividing tumor cells (Klenow (1963) Biochem. Biophys. Acta. 76:347-53; Muller et al. (1997) Cancer Research 37:3824-33). As an antiviral agent, 3′-deoxyATP exhibits its biological activity by directly inhibiting viral replication through its ability to block polyadenylation, thus interfering with processing and maturation of viral and host mRNA.

The effectiveness of Cordycepin as a drug is severely compromised by rapid deamination to the inactive metabolite 3′-deoxyinosine. This reaction is catalyzed by adenosine deaminase (“ADA”), an enzyme which is abundant in a wide range of tissues [Agarwal et al. (1975) Biochem. Pharmacol. 24:693-701). In order to prevent deamination of Cordycepin or other adenosine nucleoside analogs, coadministration of ADA inhibitors such a 2′-coformycin or 2′-deoxycoformycin is necessary to achieve significantly effective concentrations of nucleoside analog both in vitro and in vivo [Johns et al. (1976) Biochem. Pharmacol. 25: 441-4; Adamson et al. (1977) Pharmacology 15:84-89; Koc Y et al. (1996) Leukemia 10:1019-24; Sugar et al. (1998) Antimicrobial Agents and Chemotherapy 42(6): 1424-7; Kodama et al. (2000) BioChem Pharmacol. 59(3):273-81). Unfortunately, these ADA inhibitors are rather cytotoxic, as they inhibit ADA so efficiently that enzyme resynthesis is required to regenerate its activity (Padua et al. (1992) J. Neurochem. 58:421-9; Agarwal et al. (1977) Biochem. Pharmacol. 26:359-67).

In an effort to seek an effective alternative to the use of ADA inhibitors, several Cordycepin prodrugs have been developed that are protected from inactivation by deamination while retaining the potent biological activity of the parent drug. 3′-deoxyadenosine N′-oxide (3′-dANO) (Svendson et al. (1992) Cancer Chemother. Pharmacol. 30: 86-94) and 9-(β-D-arabinofuranosyl)-6-azidopurine (6-AAP) [Kotra et al. (1998) J. Med. Chem.; U.S. Pat. No. 6,271,212) are examples of Cordycepin prodrugs in which the amine group of Cordycepin is replaced with a small functional group (N-oxide or Azido). These agents are metabolically inert until they enter a target cell that is capable of reducing the prodrug to regenerate the metabolically active Cordycepin. These prodrugs provide only a brief extension to the half-life of Cordycepin and thus there remains a significant need in the art for nucleoside analogs with improved pharmacokinetic profiles.

### SUMMARY OF THE INVENTION

The present invention provides compounds having increased resistance to inactivation by metabolic enzymes and, thus, improved therapeutic efficacy and bioavailability. Particular compounds of the invention include nucleotide and nucleoside analogs, wherein the free amine is derivatized with one or more aminal or thioaminal groups to protect the compound from deamination by, for example, deaminases and other metabolic enzymes when administered to a subject in vivo or to a biological product ex vivo. Accordingly, compounds of the invention include a variety of therapeutic nucleotide and nucleoside analogs (e.g., prodrugs) having increased resistance to degradation and inactivation.

Compounds of the invention can be characterized as having the structure
A-Xₙ

wherein A is selected from the group consisting of a nucleotide, a nucleotide analog, a nucleoside, and a nucleoside analog, and wherein A comprises at least one amine, wherein the amine is derivatized with at least one X;

wherein n is either 1 or 2; and

wherein each X is independently selected from the group consisting of an aminal having the structure —(CR₄R₅)—O—R₆, a thioaminal having the structrure —(CR₁R₂)—S—R₃, and combinations thereof, wherein each R may be the same or different and is independently selected from the group consisting of a hydrogen, an alkyl, a substituted alkyl, an alkoxy, a substituted alkoxy, an aryl, and a substituted aryl. Accordingly, when n is 2, X can be two thioaminals, two aminals or a combination of one thioaminal and one aminal.

In various embodiments, the amine may be a primary or secondary amine. In addition, the amine may be an aromatic amine.

In one embodiment, A comprises the nucleoside analog, Cordycepin (3′-deoxyadenosine), or another adenosine analog. In other embodiments, X comprises, for example, analogs of cytosine, fluoroarabinofluorcytosine, ganciclovir, trimethoprim, penciclovir, valaciclovir, vidarabine, arabinofuranosyladenine (Ara-A), arabinocytidine, acyclovir, arabinofuranosylcytosine (Cytarabine, Ara-C), arabinofuranosyl-5-fluorocytosine, cytidine, 2′-deoxycytidine, famciclovir, flucytosine, 5-fluorocytosine, 5′-fluoro-1′,2′-dioxalane cytosine (B-D-FDOC), 5-fluoro-2′3′-dideoxycytidine (D-D-FddC), 5-fluoro-2′,3′-dideoxy-2′,3′-didehydrocytosine (B-D-Fd4C), and 5-fluoro-3′deoxy-3′thiacytidine (B-D-FTC). Such compounds also include monophosphate nucleotides, a diphosphate nucleotides, triphosphate nucleotides and analogs thereof.

In a particular embodiment, the invention provides a compound having the following structure:

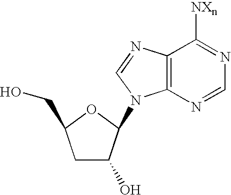

wherein n is either 1 or 2; and

wherein each X is independently selected from the group consisting of a hydrogen, an aminal having the structure —(CR₄R₅)—O—R₆, a thioaminal having the structure —(CR₁R₂)—S—R₃, and combinations thereof, wherein at least one X is the thioaminal or the aminal, wherein each R is the same or different and is independently selected from the group consisting of a hydrogen, an alkyl, a substituted alkyl, an alkoxy, a substituted alkoxy, an aryl, and a substituted aryl.

In another particular embodiment, the invention provides a compound having the following structure: (6-N-phenylthioaminal Cordycepin)

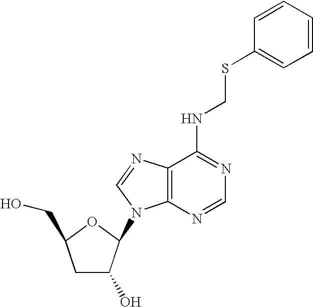

In another particular embodiment, the invention provides a compound having the following structure: (6-N-(4-methyl)phenylthioaminal Cordycepin)

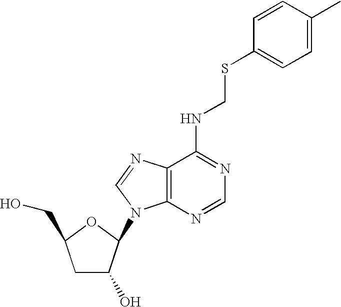

Compounds of the invention also can be modified to include one or more agents that target the compound, for example, to a particular cell or pathogen. Suitable targeting agents include, but are not limited to, antibodies, hormones, antibody fragments, aptamers, peptides, small molecules and other binding agents. Alternatively or additionally, the compounds can be modified to include moieties that increase or decrease solubility, such as polyethylene glycol (PEG), phophate esters, phosphoramide esters, amino acid esters, t-BOC amino acids, lipids, steroids, amine-containing carbon chains, amino acids, and peptides.

In another aspect, the invention provides a composition containing one or more compounds of the invention, formulated in a suitable carrier (e.g., for pharmaceutical application). The composition can further include one or more other therapeutic agents, such as an anti-cancer agent, anti-viral agent or anti-fungal agent.

In yet another aspect, the invention provides a method for protecting a compound (e.g., from deactivation e.g., by a deaminase) comprising at least one amine (e.g., a primary amine, a secondary amine and/or an aromatic amine), by derivatizing the amine with at least one substituent, wherein the substituent is selected from the group consisting of a thioaminal having the structure —(CR₁R₂)—S—R₃, an aminal having the structure —(CR₄R₅)—O—R₆, and combinations thereof, wherein each R is the same or different and is independently selected from the group consisting of a hydrogen, an alkyl, a substituted alkyl, an alkoxy, a substituted alkoxy, an aryl, and a substituted aryl.

In another aspect, the invention provides a method for protecting an amine, comprising derivatizing the amine with at least one substituent selected from the group consisting of an aminal, a thioaminal, or a combination thereof. In yet another aspect, the invention provides a method for protecting a nucleoside, a nucleotide or an analog thereof, wherein the nucleoside, nucleotide or analog comprises at least one amine, comprising derivatizing the amine with at least one substituent selected from the group consisting of an aminal, a thioaminal, or a combination thereof. In various embodiments, the thioaminal in the preceding aspects can comprise the structure —(CR₁R₂)—S—R₃, wherein each R is the same or different and is independently selected from the group consisting of a hydrogen, an alkyl, a substituted alkyl, an alkoxy, a substituted alkoxy, an aryl, and a substituted aryl. In certain embodiments, the aminal in the preceding aspects can comprise the structure —(CR₄R₅)—O—R₆, wherein each R is the same or different and is independently selected from the group consisting of a hydrogen, an alkyl, a substituted alkyl, an alkoxy, a substituted alkoxy, an aryl, and a substituted aryl.

Such protection can prolong the half-life of the compound, increase its bioavailability and/or increase its stability. Accordingly, the method of the invention can be used in a variety of treatments to improve the efficacy of compounds, and in the context of, for example, RNA interference and antisense modulation. In particular, nucleotide and nucleoside analogs of the invention having increased resistance to deamination can be incorporated into short interfering RNA molecules and antisense molecules to improve the efficacy of the molecules in blocking gene expression.

In yet another aspect, the invention provides for a nucleotide, nucleoside or analog thereof, wherein the nucleotide, nucleoside or analog comprises at least one amine, and wherein the amine is derivatized with at least one substituent selected from the group consisting of an aminal, a thioaminal, or a combination thereof.

Compounds of the present invention can be used in a variety of therapeutic applications, including applications in which the parent compounds from which they are derived are used. For example, derivatized nucleotide and nucleoside analogs of the invention can be used in the treatment of cancers and parasitic, viral and fungal infections. In such applications, the compounds can be administered to a subject in vivo or to biological or food products ex vivo.

Particular neoplastic disorders that can be treated include, for example, leukemias, lymphomas, sarcomas, carcinomas, neural cell tumors, squamous cell carcinomas, germ cell tumors, undifferentiated tumors, seminoma, melanomas, neuroblastomas, mixed cell tumors, metastatic neoplasias, terminal deoxynucleotidyl transferase-positive leukemias, or terminal deoxynucleotidyl transferase-positive lymphomas, and neoplasias due to pathogenic infections and malignancy.

Particular parasitic infections that can be treated include, for example, infections by trypanasonal parasites, such as trypanasonal brucei and trypanasonal cruzi, and infections by plasmodium parasites, such as plasmodium falciparum, plasmodium vivax, plasmodium ovale and plasmodium malarie.

Particular fungal infections that can be treated include, for example, infections by Candida krusei, C. glabrata, C. albicans and C. tropicalis, C. parapsilosis, Trichophyton rubrum, T. mentagrophytes, T. tonsurans, Microsporum audouini, M. canis and T. floccosum, Nocardia asteroides and N. brasiliensis, Actinomyces israelii, species of the genera Mucor, Absidia, Rhizopus, Cunninghamella, Zygomycetes, Aspergillus fumigatus, A. flavus, A. niger, species of the genera Aspergillus, Crypttococcus neoformans, Paracoccidioides brasiliensis, Coccidioides immitis, Blastomycetes dermatitis and Histoplasma capsulatum.

Other embodiments of the invention will be clear from the following detailed description and also are intended to be included within the scope of the invention.

### DETAILED DESCRIPTION OF THE INVENTION

The present invention is based, in part, on the discovery that by derivatizing compounds containing the free amines, such as nucleotides, nucleosides and analogs thereof, with aminal and thioaminal groups, resistance to enzymatic deamination of the compound is substantially increased until non-enzymatic hydrolysis (e.g., in vivo) regenerates the active molecule. Accordingly, derivatized compounds of the present invention are protected until they are selectively hydrolyzed to their active amine counterparts. As a result, the compounds of the present invention have a significantly extended half-life.

Definitions

As used herein, the following terms shall have the definitions referred to below.

“Free amine” shall refer to an amine substituent (e.g., a primary aryl amine) that is capable of being deaminated by a deaminase. The amine may be, but is not limited to, a primary amine or a secondary amine. In addition, the amine may be an aromatic amine.

“Alkyl” shall mean a straight, branched, or cyclic alkyl group having at least one carbon atom such as methyl, ethyl, propyl, isopropyl, butyl, sec-butyl, t-butyl, and pentyl groups.

“Alkoxy” shall denote an alkyl group as described above bonded through an oxygen linkage (—O—). Examples of alkoxy groups include methoxy, ethoxy, propoxy, isopropoxy, butoxy, and t-butoxy groups.

“Aryl” refers to monocyclic or bicyclic aromatic rings, such as phenyl, substituted phenyl, and the like. An aryl group contains at least one ring having at least 5 atoms with alternating (resonating) double bonds between adjacent carbon atoms or suitable heteroatoms.

Optionally substituted alkyl groups, optionally substituted alkoxy groups, and optionally substituted aryl groups may bear one or more substituents including, but not limited to halogen, hydroxyl, amino, alkylamino, dialkylamino, carboxyl, mercapto, nitro, alkoxycarbonyl, aminocarbonyl, alkylaminocarbonyl, dialkylaminocarbonyl, alkylcarbonylamino, alkylcarbonyl(alkyl)amino, sulphate, and phosphate substituents. As used herein, the term “halogen” includes fluorine, chlorine, bromine, or iodine.

The term “targeting agent” as referred to herein includes moieties and compounds that can be linked to a compound of the invention to direct or “target” the compound to, for example, a target cell or pathogen. Suitable targeting agents are well known in the art and include, for example, antibodies, binding peptides, cellular ligands and small molecules. Targeting agents also include liposomes and other carrier molecules that, for example, can encapsulate the compound. The term “antibody” as referred to herein includes whole antibodies and any antigen binding portion or single chain thereof. An “antibody” refers to a glycoprotein comprising at least two heavy (H) chains and two light (L) chains inter-connected by disulfide bonds, or an antigen binding portion thereof. The term “antigen-binding portion” of an antibody (or simply “antibody portion”), as used herein, refers to one or more fragments of an antibody that retain the ability to specifically bind to an antigen. Examples of binding fragments include a Fab fragment, a F(ab′)₂ fragment, a Fd fragment, a Fv fragment, and a dAb fragment.

Derivatized Compounds

In one aspect, the invention provides a compound having the general structure A-Xₙ, wherein A is selected from the group consisting of a nucleotide, a nucleotide analog, a nucleoside, and a nucleoside analog, and wherein A comprises at least one amine that is derivatized by X which can be a thioaminal having the structure —(CR₁R₂)—S—R₃ or an aminal having the structure —(CR₄R₅)—O—R₆ or both. N can be 1 or 2. Each R can be the same or different from another R. Each R is independently a hydrogen, an alkyl, a substituted alkyl, an alkoxy, a substituted alkoxy, an aryl, or a substituted aryl. Aminal and thioaminal derivatives of the invention include the free forms as well as salts (e.g., pharmaceutically acceptable salts) resulting form their being formulated with, for example, hydrochloride, hydrobromide or sulfate salts; organic acid addition salts such as citrate, acetate, or oxalate; or salts derived from inorganic bases including alkali metal salts such as sodium salt or alkaline earth metal salts such as magnesium salt.

In one embodiment of the present invention, A is a nucleotide, nucleotide analog, a nucleoside, or a nucleoside analog. Accordingly, A can be a purine or a pyrimidine. A also can be a polynucleotide, a polynucleoside or an analog thereof. A also can be a monophosphate, diphosphosphate or triphosphate of a nucleotide, a nucleoside or an analog thereof. In a particular embodiment, A is Cordecypin. In other particular embodiments, A is adenosine, cytosine, arabinofuranosyladenine (Ara-A), arabinocytidine, acyclovir, arabinofuranosylcytosine (Cytarabine, Ara-C), arabinofuranosyl-5-fluorocytosine, cytidine, 2′-deoxycytidine, famciclovir, flucytosine, 5-fluorocytosine, 5-fluoro-1′,2′-dioxalane cytosine (B-D-FDOC), 5-fluoro-2′3′-dideoxycytidine (D-D-FddC), 5-fluoro-2′,3′-dideoxy-2′,3′-didehydrocytosine (B-D-Fd4C), 5-fluoro-3′deoxy-3′thiacytidine (B-D-FTC), fluoroarabinoflurocytosine, ganciclovir, trimethoprim, penciclovir, valaciclovir, and vidarabine. A also can be a monophosphate, diphosphate or and triphosphate of the aforementioned nucleoside analogs.

Particular Cordycepin derivatives of the invention include those having the formula: 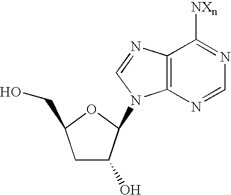
wherein n is either 1 or 2, and X is selected from the group consisting of a thioaminal having the structure —(CR₁R₂)—S—R₃, an aminal having the structure —(CR₄R₅)—O—R₆, and combinations thereof, and R is selected from the group consisting of a hydrogen, an alkyl, a substituted alkyl, an alkoxy, a substituted alkoxy, an aryl, and a substituted aryl.

Other particular Cordycepin derivatives of the invention include substituted phenylthioaminals of Cordycepin having the following structures:

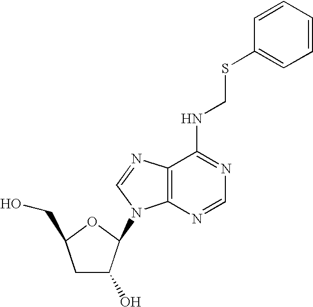

### 6-N-phenylthioaminal Cordycepin

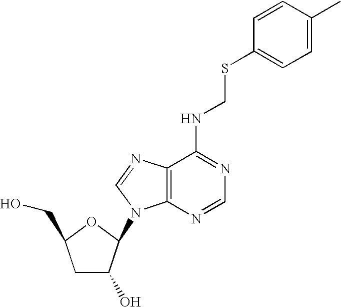

### 6-N-(4-methyl)phenylthioaminal Cordycepin

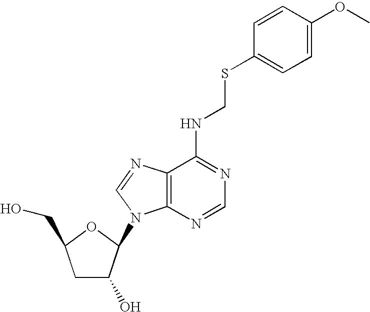

### 6-N-(4-methoxy)phenylthioaminal Cordycepin

The nucleoside and nucleotide analogs described above may contain more than one amine. In such circumstances, all or less than all of the amine groups may be substituted with aminal and/or thioaminal substituents. In cases where n is 2, each amine can be derivatized with two aminals, one aminal and one thioaminal, or two thioaminals (i.e., resulting in a bis-thioaminal). Where the resulting compound is a bis-thioaminal, the two thioaminals may the be the same or may be different.

In one embodiment the amine may be a primary or a secondary amine. In addition, the amine may be an aromatic amine.

In one embodiment, X is a thioaminal having the structure —(CR₁R₂)—S—R₃ or an aminal having the structure —(CR₄R₅)—O—R₆ wherein m is any number between 1 and wherein R is selected from the group consisting of a hydrogen, an alkyl, a substituted alkyl, an alkoxy, a substituted alkoxy, an aryl, and a substituted aryl.

Compounds of the present invention also can be modified to alter the functionality, solubility and/or cell permeability of the compound. For example, additional moieties or subsituents may be incorporated within the compound (e.g, to the thioaminal group, the aminal group or directly to the nucleoside, nucleotide or analog thereof), as is well known in the art, to modulate (e.g., increase or decrease) the solubility of the compounds. Such modifying agents include, for example, polyethylene glycol (PEG), phosphate esters, phosphoramidate esters, lipids, steroids, amine-containing carbon chains, amino acids, amino acid esters (e.g., t-BOC amino acids) and peptides.

Further modifications encompassed by the invention include linking the compounds to a targeting agent that binds to a target cell or pathogen. Suitable targeting agents include, but are not limited to, antibodies, aptamers, hormones, binding peptides, liposomes, cellular ligands and small molecules. Such targeting agents can be naturally occurring molecules, recombinantly produced molecules, or engineered molecules. For example, the molecule can be an engineered protein containing non-peptidic components, such as a small organic moiety, sugar residues, or RNA.

Lipophilic groups also can be attached to compound (e.g., to an R group of the aminal or thioaminal substituents) to increase cell permeability and increase the uptake of the compound into target cells. Alternatively or additionally, the compound can be encapsulated into liposomes or other microcapsules, as is well known in the art.

Compounds of the present invention can be formulated as a composition (e.g., a pharmaceutical composition) along with a suitable carrier. Suitable carriers are well known in the art and include, for example, agents that facilitate administration, increase physiological stability, facilitate storage or increase half-life of the composition. Accordingly, in another embodiment, the invention provides a composition comprising at least one derivatized compound and a suitable (e.g., pharmaceutically acceptable) carrier. Examples of suitable carriers known in the art include, but are not limited to, sucrose, lactose, starch, water, salt (e.g., salt solutions), alcohol, oils (e.g., vegetable oils), polyethylene glycols, glycerols, collagen, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, saccharides, polysaccharides, and combinations and modifications of these substances.

Additionally, the compositions can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. Compositions of the invention may optionally include flavoring agents and other agents that may be necessary or desirable to increase shelf-life, such as preservatives, anti-oxidants and other components advantageous for manufacture and distribution of the composition.

Methods for formulating pharmaceutical reagents are well known in the art, and the amount of active compound in the composition can be readily determined by those of ordinary skill depending, for example, on the nature and size of the subject or product to be treated. In one embodiment, the derivatized compound of the invention is added at a concentration of between about 0.01 mM to about 20 mM (e.g., between about 1.0 mM to about 10 mM). The compositions also can contain concentrated forms of the active ingredient. In cases where the composition is formulated as a liquid (vol./vol.) or a solid (wt./wt.), the composition can contain active components at between about 0.001% to about 100%, between about 0.01% to about 10.0%, and between about 0.1% to about 5.0%. The compositions can also be further diluted, if necessary, to prolong direct contact with skin or other bodily tissues.

Methods of Manufacture

Derivatized compounds of the invention can be prepared using techniques well known in the art. In particular, aminal derivatives can be synthesized, for example, by reductive alkylation of the free amine of a nucleoside, nucleotide or analog thereof with a corresponding alcohol (R′-OH) (e.g., alkylalcohol, arylalcohol, or alkoxyalcohol) in the presence of formaldehyde, cyanoborohydride and a suitable organic solvent (Borch et al. (1972) J. Org. Chem. 37(10):1673-4). For example, an aminal prodrug of the nucleoside analog Cordycepin may be prepared according to the following scheme:

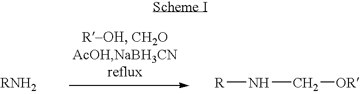

Thioaminal derivatives may be synthesized by methods known in the art. For example, thioaminal derivates may also be synthesized by reductive alkylation of the free amine of a nucleotide, nucleoside or analog thereof with a corresponding thiol (R′-SH) (e.g., alkylthiol, arylthiol, or alkoxythiol) in the presence of formaldehyde and glacial acetic acid in ethanol (Kemal et al. (1980) Synthesis 1025-8) according to the following scheme:

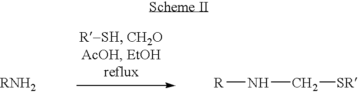

Bis-thioaminal derivatives may be synthesized in the same fashion as thioaminals, with the exception that the solvent 2,2,2-trifluoroethanol replaces the ethanol according to the following scheme.

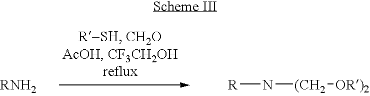

Other techniques known in the art may be used to prepare the aminal and thioaminal derivatives of the present invention.

Therapeutic Uses

Derivatized compounds (e.g., prodrugs) of the present invention regenerate to the parent nucleoside, nucleotide, or analog thereof upon non-enzymatic hydrolysis (e.g., when placed in vivo). As a result, the compounds can be used to a variety of diseases treatable by the parent nucleoside, nucleotide, or analog thereof. Alternatively or additionally, the compounds can be used to in the context of RNA interference and antisense modulation, as is known in the art for underivatized nucleotide and nucleoside analogs.

Treatment of Parasitic Infections

In one embodiment, the present invention provides a method for treating a subject or a biological product infected with a parasite, including administering to the subject or biological product a therapeutically effective amount of a compound having the structure A-Xₙ, where A is a nucleotide, a nucleotide analog, a nucleoside, or a nucleoside analog, and where A has at least one amine. N is either 1 or 2. X is a thioaminal having the structure —(CR₁R₂)—S—R₃ or an aminal having the structure —(CR₄R₅)—O—R₆. R is a hydrogen, an alkyl, a substituted alkyl, an alkoxy, a substituted alkoxy, an aryl, or a substituted aryl.

Compositions and methods of the invention are effective against a wide variety of parasites. Parasites are generally transferred by insects, in which essential parasite life cycles occur. Parasites may be transmitted to humans directly from insects or indirectly from insects through another animal host that acquired the parasite. Generally parasitic diseases can infect the blood, the tissues, the lymphatic system, major organs and organ systems, the dermis and the gastrointestinal tract. Examples of parasitic diseases characterized by infections of the blood, lymph and tissues, include, but are not limited to trypanosomiasis, leishmania, toxoplasmosis, sarcocystis, pneumocystis, schistosomiasis and elephantitis. Gastrointestinal disorders include, but are not limited to, Entamoeba, the flagellates Giardia lamblia, Dientamoeba fragilis and Trichomonas vaginalis, the nematodes Ancyclostoma, Ascaris, Enterobius and both cutaneous and visceral leishmaniasis. Examples of parasites include, but are not limited to, species of the genera Entamoeba, Ascaris, Ancyclostoma, Strongyloides, Trichuris, Wuchereria, Leishmania, Plasmodium, Toxoplasma, Sarcocystis, Pneumocystis, Schistosoma, Loa, Onchocerca, Brugia, Dipetalonema, Mansonella, Dracunculus, Babesia and Trypanosoma. In particular, Trypanosmiasis is associated with trypanasonal parasite (such as trypanasonal brucei and trypanasonal cruzi) and malaria is associated with plasmodium parasite (such as plasmodium falciparum, plasmodium vivax, plasmodium ovale and plasmodium malarie).

The compositions of the present invention are administered to a subject or biological product for a necessary period of time to achieve the desired result. Subjects with a suspected or diagnosed parasitic infections may only require treatments for short periods of time or until the infection has proceeded to remission or has been effectively eliminated. Alternatively, to effectively eliminate certain parasites, administration may require long term treatments such as for months or years.

Administration may be by any of the methods of administration described herein. Nasal sprays are a preferable method for administering compositions to the pulmonary system and the bloodstream and are useful to treat diseases caused by species of the genera Trypanosoma, Leishmania, Plasmodium and Schistosoma. Access to the gastrointestinal tract is also achievable using oral, enema, or injectable forms of administration. Such forms of compositions may also be useful to treat gastrointestinal disorders as described herein. Oral administration may often be the most effective method to administer compositions directed to parasites of the gastrointestinal tract such as infections by Entamoeba histolytica, E. coli, E. poleki, Ascaris lumbricoides, Giardia lamblia, Enterobius vermicularis, Necator americanus, Wuchereria bancrofti and various species of Ancyclostoma.

In another embodiment of the invention, the composition contains additional therapeutic agents to maximize the effect of the compositions in an additive or synergistic manner. Agents which may be effective in combination with the compositions of the invention include other drugs and treatments which are known or suspected to have a positive effect against the parasite. Examples of additional agents known to be effective against one or more pathogenic parasites include benzidazoles, nitrofurfurylidines, dimercaprols, suramins, pentamidines, melarsoprols, melarsen oxides, quinines, sulfonamides, sulfones, chloroquines, pyrimethamines, antimony sodium gluconates, sulfadiazines, and derivatives, modifications and combinations of these agents. Therapies using various combinations of these agents would be safe and effective therapies against infections. Combinations of therapies may also be effective in inducing suppression or elimination of an infection such as compositions of the invention plus radiation therapy, toxin or drug conjugated antibody therapy using monoclonal or polyclonal antibodies directed against, for example, the parasite, infected cells, gene therapy or specific anti-sense therapy. Effects may be additive, logarithmic, or synergistic, and methods involving combinations of therapies may be simultaneous protocols, intermittent protocols or protocols which are empirically determined.

Treatment of Fungal Infections

In another embodiment, the present invention provides a method for treating a subject or a biological product infected with a fungal or fungal-like organism, including administering to the subject or biological product a therapeutically effective amount of a compound having structure A-Xₙ, where A is a nucleotide, a nucleotide analog, a nucleoside, or a nucleoside analog, and where A has at least one amine. N is either 1 or 2. X is a thioaminal having the structure —(CR₁R₂)—S—R₃ or an aminal having the structure —(CR₄R₅)—O—R₆. R is a hydrogen, an alkyl, a substituted alkyl, an alkoxy, a substituted alkoxy, an aryl, or a substituted aryl.

Compounds of the invention are effective against a wide variety of fungal and fungal-like organisms. Examples of infections which can be treated by compounds of the invention include the fungal diseases candidiasis, tinea pedis, tinea corporis and tinea capitis, aspergillosis, mucormycosis, phaeohyphomycosis, cryptococcosis, coccidioidomycosis, blastomycosis, histomycosis, paracoccidiodomycosis and the Dematiaceous infections, and the fungal-like diseases nocardiosis and actinomycosis.

Treatable infections can occur in blood, tissues, the lymphatic system, the respiratory and gastrointestinal tract, the major organs and organ systems, and the dermis. Fungal organisms which are sensitive to treatments include Candida krusei, C. glabrata, C. albicans and C. tropicalis, the causative agents of candidiasis, and Trichophyton rubrum, T. mentagrophytes, T. tonsurans, Microsporum audouini, M. canis and T. floccosum, the causative agents of tinea pedis, tinea corporis and tinea capitis. Compounds can also be used to treat infections by Nocardia asteroides and N. brasiliensis, Actinomyces israelii, species of the genera Mucor, Absidia, Rhizopus, Cunninghamella and unrelated Mucorales, Aspergillus fumigatus, A. flavus, A. niger and other species of Aspergillus, Cryptococcus neoformans, Paracoccidioides brasiliensis, Coccidioides immitis, Blastomycetes dermatitis and Histoplasma capsulatum. In addition, diseases which can be treated include, but are not limited to, mycotic or mycotic-like infections of the blood, lymph and tissues such as candidiasis, aspergillosis, mucormycosis, blastomycosis, cryptococcosis, blastomycosis, histoplasmosis, coccidioidomycosis and paracoccidiodo-mycosis, nocardiosis and actinomycosis.

Compositions of the invention can further include other anti-fungal agents. Examples of such additional agents known to be effective against one or more pathogenic fungal and fungal-like organisms include flucytosine, mycoconazole, fluconazole, itraconazole, ketoconazole and griseofulvin, antibiotics such as amphotericin B, sulfadiazine, penicillin, chlortetracycline, chloramphenicol, streptomycin and other sulfonamides, and derivatives, modifications and combinations of these agents. Therapies using various combinations of these agents would be safe and effective therapies against infections. Combinations of therapies may also be effective in inducing suppression or elimination of an infection such as compositions of the invention plus radiation therapy, toxin or drug conjugated antibody therapy using monoclonal or polyclonal antibodies directed against, for example, the organism, infected cells, gene therapy or specific anti-sense therapy. Effects may be additive, logarithmic, or synergistic, and methods involving combinations of therapies may be simultaneous protocols, intermittent protocols or protocols which are empirically determined.

Treatment of Neoplastic Disorders

In another embodiment, the present invention provides a method for treating a subject or a biological product infected having a neoplastic disorder, including administering to the subject or biological product a therapeutically effective amount of a compound having the structure A-Xₙ, where A is a nucleotide, a nucleotide analog, a nucleoside, or a nucleoside analog, and where A has at least one amine. N is either 1 or 2. X is a thioaminal having the structure —(CR₁R₂)—S—R₃ or an aminal having the structure —(CR₄R₅)—O—R₆. R is a hydrogen, an alkyl, a substituted alkyl, an alkoxy, a substituted alkoxy, an aryl, or a substituted aryl.

The neoplastic disorder may be any disease that can be characterized as a neoplasm, a tumor, a malignancy, a cancer or a disease which results in a relatively autonomous growth of cells. The neoplastic disorder may be a leukemia, lymphoma, sarcoma, carcinoma, neural cell tumor, squamous cell carcinoma, germ cell tumor, undifferentiated tumor, seminoma, melanoma, neuroblastoma, mixed cell tumor, metastatic neoplasia, neoplasia due to pathogenic infections or other malignancy.

As with the embodiments described above, the compound can be formulated as a composition and may contain additional therapeutic agents, such as anti-cancer agents. Such therapeutic agents include, for example, a chemotherapeutic agent, an alkylating agent, a purine or pyrimidine analog, a vinca or vinca-like alkaloid, an etoposide or etoposide-like drug, an antibiotic, a corticosteroid, a nitrosourea, an antimetabolite, a platinum based cytotoxic drug, a hormonal antagonist, an anti-androgen, an anti-estrogen, or a derivative, modification or combination of these agents.

Therapeutic Administration

Compounds of the present invention can be administered to a variety of subjects in vivo, or to biological or food products ex vivo, e.g., to remove pathological contaminants. Subjects that may be treated include both human and non-human subjects. Suitable subjects include, but are not limited to, mammals such as a human, dog, cat, horse, cow, cattle, pig, sheep, goat, rodent, camel, chicken, or wild animal. For example, since parasitic infections are often transmitted to humans indirectly from animals, treatment of animals is envisioned. Zoo animals such as monkeys (primates) tend to acquire parasitic infections which are treatable with compositions of the invention. Elimination of the parasite in the animal host is an effective means for eliminating or preventing infections in humans. In addition, since most fungal organisms can infect across species and genera boundaries, elimination of the fungal organisms in the animal is sometimes an effective means for eliminating or preventing infections in humans. Zoo animals, such as penguins and monkeys, are typically infected with a range of different fungal organisms and may be suitable subjects for the methods of the present invention.

Administration may be to an adult, an adolescent, a child, a neonate or an infant, or even to a subject in utero. Dosages range from between about 1 ng/kg subject weight to about 50 mg/kg subject weight. Administration of the composition may be for a short term, continuous or sporadic as necessary. Alternatively, administration may be for a long term ranging from months to years. As compositions of the invention are generally safe and non-toxic at required dosages, this does not present a problem.

Compositions are administered in a manner which is most useful for the infection being treated. Useful methods of administration include oral, parenteral, sublingual, rectal or enteral administration, pulmonary absorption or topical application. Parenteral administration may be intravenously, subcutaneously, intrapleurally, intracavitarily, intramuscularly, intra-arterially, intrathecally, intraperitoneally or by direct injection or other administration directly to the site or sites of infection. Injectable forms of administration are sometimes preferred for maximal systemic effect. When long term administration by injection is necessary, medi-ports, in-dwelling catheters, or automatic pumping mechanisms may be used. These devices provide direct and immediate access to the arteries in and around the heart and other major organs and organ systems. For example, such devices are useful for treating parasitic diseases that infect organs and organ systems such as the blood and tissue dwelling nematodes, malaria, trypanosomiasis and leishmania.

Another effective method of administering compositions (e.g., to infectious sites) may be by transdermal transfusion such as with a transdermal patch and other means of direct contact with affected tissues, or by administration (e.g., to an internal infection) through an incision or some other natural or artificial opening into the body. Compositions may also be administered to the nasal passages as a spray. Diseases localized to the respiratory tract, the head and brain area are treatable in this fashion as arteries of the nasal area provide a rapid and efficient access to the upper areas of the body. Sprays also provide immediate access to the pulmonary system and the bloodstream. Compositions may be administered as a bolus injection or spray, or administered sequentially over time (episodically) such as every two, four, six or eight hours, every day (QD) or every other day (QOD), or over longer periods of time as necessary (e.g., weeks to months or for as long as it takes an infection to resolve or for the subject's own system to be able to overcome an infection).

Orally active compositions are preferred as oral administration is usually the safest, most convenient and economical mode of drug delivery. Oral administration can be disadvantageous because compositions are poorly absorbed through the gastrointestinal lining. Compounds which are poorly absorbed tend to be highly polar. Consequently, compounds which are effective, as described herein, may be made orally bioavailable by reducing or eliminating their polarity without significantly compromising their functional activity. This can often be accomplished by formulating a composition with a complimentary reagent which neutralizes its polarity, or modifying the compound with a neutralizing chemical group. Oral bioavailability is also a problem because drugs may be exposed to the extremes of gastric pH and gastric enzymes. These problems can be overcome in a similar manner by modifying the molecular structure to be able to withstand very low pH conditions and resist the enzymes of the gastric mucosa such as by neutralizing an ionic group, by covalently bonding an ionic interaction, or by stabilizing or removing a disulfide bond or other relatively labile bond.

When the composition is administered orally, it may be in the form of a liquid, a spray, a powder, a pill, a tablet or a capsule. To facilitate oral administration, compositions of the invention will preferably include flavoring agents and other agents to increase shelf-life.

Administration by any method can be accurately quantitated by measuring levels of the composition from a sample of bodily fluid such as blood, serum or plasma. Effective serum levels of active components of the invention are between about 0.01 nM to about 50 mM. When applied by direct contact, effective levels of active ingredient may sometimes be analyzed by determining concentration of the composition in the areas which are in close contact with the area of application. For example, when applied topically to the skin, effective levels may be determined from fluid or tissue samples of the dermal tissues within a few centimeters under the area of application. In such cases, composition strength may be predetermined and used as a concentrated solution.

Compositions can be administered by oral or enema formulations, or by rectal irrigation to maximize their contact with and effectiveness on the gastrointestinal system. In such cases, dosages are between about 1% to about 20% (vol/vol.) or between about 1 mM to about 100 mM. Doses are administered until symptoms improve sufficiently for the subject's immune system to resolve the infection (e.g., parasitic or fungal) or the parasite is killed or eliminated. Multiple and frequent dosing is not problematic because the compounds of the invention are safe, non-toxic and physiologically stable.

Positive effects of treatment include a reduction of parasite, parasitemia, or fungal (or fungal-like) organism load; death or inactivation of the parasite or the fungal (or fungal-like) organism; decreased infectivity of the parasite; decreased infectivity or spore-forming ability of the fungal or fungal-like organism; or elimination of the parasite or fungal (or fungal-like) organism from the body. Preferably, the subject has a parasitemia or infection which is reduced at least 100-fold, more preferably 1000-fold, and even more preferably is undetectable after treatment. Parasitemia or infection may be determined by growing parasites or organisms, respectively, from biological samples obtained from the subject suspected to be infected with the parasite or organism into suitable cultures and counting the parasites or colonies which can be grown. Alternatively, biological samples are obtained from selected areas of the subject suspected to be infected and the numbers of parasites or organisms visualized directly or indirectly under a microscope or other suitable devise and counted. Fluorescent-conjugated antibodies may also be used in, for example, an ELISA or other markers to detect mycotic or related antigen or anti-antigen antibodies or parasitic antigen or anti-antigen antibodies in a biological sample to determine the degree of infection and the effect of treatments.

Another embodiment of the invention is directed to compounds described above which can be used prophylactically. For example, subjects exposed to areas where a parasitic, fungal or fungal-like disease is endemic may be continuously treated with compositions to prevent a parasitic, fungal or fungal-like infection from taking hold. Subjects who have been genetically screened and determined to be at high risk for the future development of an infection may also be administered compounds of the present invention, possibly beginning at birth and possibly for life. Administration may be by any means described and dosages may be reduced in comparison to dosages required for treatment. Both prophylactic and therapeutic uses are readily acceptable because these compounds are generally safe and non-toxic at useful dosages.

Another embodiment of the invention is directed to methods for the treatment of biological products (e.g., suspected of being contaminated with a parasite). Products which can be treated or pretreated include, but are not limited to, whole blood, fractionated blood, plasma, serum, transplantable organs, living cells including bone marrow, stem cells, primary cells surgically obtained and established cell lines, and products derived from living cells. Products which can be derived from living cells include blood products such as insulin, the blood clotting factors (e.g., Factor V, VIII, VIII, IX, X, XI, XII), cytokines (e.g., interferon α, β, or γ, the interleukins II-1, II-2, II-3, etc.), complement proteins, antibodies, immune system regulators, recombinant proteins and other macromolecular products.

Treatment can involve contact of the biological product with a solution comprising a compound of the present invention. Products may be sprayed, powdered, sprinkled, misted, subjected to pressurizing conditions, submerged, coated or otherwise administered compounds of the invention to foster contact between the compound and the parasite, fungal organism or fungal-like organism. Contact may also be encouraged by incubating compounds of the invention with the product. Incubations may be performed at between about 0° C. to about 50° C., between about 4° C. and about 37° C., and, in a particular embodiment, at about room temperature (18°-22° C.). For example, the biological product, which may be living, is placed in a sterile container and sprayed or immersed in a solution or spied with a powder containing a derivative compound at an effective concentration. The product is maintained in this solution for a period of time as necessary to achieve the desired result (e.g., effectively inactivate or destroy the parasite or the infectious organism). This time period may be minutes to weeks, between about one minute to one week, and between about one hour to one day. The product is then removed from the solution, washed if necessary, and utilized as desired. As compositions of the invention are generally safe and non-toxic, removal of product may not even be necessary, washing may not be necessary and the product may even be stored or shipped in the composition. In such cases, compositions of the invention may also contain additional components useful or desirable to accommodate the product during storage or shipping.

For example, biological products such as blood and blood products are required in vast quantities world-wide including areas of the world where parasitic, fungal or fungal-like diseases are endemic. Food and food products (salts and spices, sugar, molasses, sorghum, alimentary paste, dairy products, oils) including grains and vegetables (corn, wheat, rice, barley, peas, soybeans), breads, fruits (grapes, citrus fruits, bananas, apples, pears) and even fish and meats can be similarly treated. Maintaining an effective quantity of a compound exhibiting anti-parasitic or anti-fungal activity in such supplies may prevent the spread of parasitic, fungal or fungal-like diseases from such products. Other products which are also required for medical uses include bone marrow and transplantable organs. Such products are typically obtained locally under emergency conditions. In such cases, there may be an undetected infection that would be passed to the subject receiving the product. Prophylactic treatment of these products would alleviate this risk and, as compositions of the invention are effective against a broad range of parasites, treatment only requires contact with this one composition. Multiple treatments would not be required or could be significantly reduced, increasing the overall chances of success for the therapy being administered to the subject.

The following examples are expected to be illustrative of the invention and in no way limit the scope of the invention:

### EXAMPLE 1

### Synthesis of Thioaminal Prodrugs of Cordycepin

A solution of Cordycepin (1 eq), and thiol 1a-9a (3-4 eq) 0.1 ml of aqueous formaldehyde (37% w/v, 2.8-3.6 eq.) and glacial acetic acid in ethanol was heated under reflux for overnight. The products were concentrated under reduced pressure and chromatographed on a silica gel and eluted with CH₂Cl₂/Methanol (8.5:1.5 v/v). Evaporation of the appropriate fractions afforded Cordycepin prodrug compounds 1b-9b as white power. 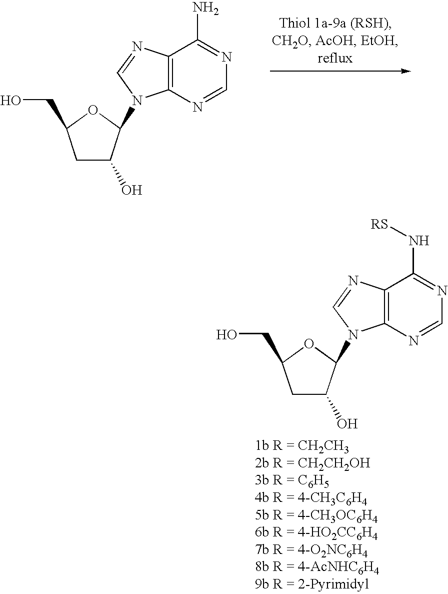
1a Ethanethiol; 1b 6-N-(Ethanethio)methyo-Cordycepin, Cordy-104 (129 mg, 37%)

¹H-NMR (300 MHz, DMSO-D₆): δ 1.19 (t, J=7.5 Hz, 3H), 1.89 (m, 1H), 1.92 (m, 1H), 2.63 (q, J=7.2 Hz, 2H), 3.50 (dd, J=12 Hz, J=3 Hz, 1H), 3.67(dd, J=12 Hz, J=3 Hz, 1H), 4.36 (m, 1H), 4.56 (bs, 1H), 4.75(s, 1.5H), 5.90(d, J=6.3 Hz, 1H), 8.26(s, 1H), 8.42 (s, 1H). FAB-HRMS calcd for C₁₃H₁₉N₅O₃S (MH+) 326.1297, found 326.1287.

2a 2-mercaptoethanol; 2b 6-N-(2-hydroxyethanethio)methyl-Cordycepin, Cordy-105 (135 mg, 38%).

¹H-NMR (300 MHz, EtOD): δ 1.13 (s, 3H), 2.06 (m, 1H), 2.38(m, 1H), 2.81(t, J=6 Hz, 2H), 3.58(s, 1.83H), 3.66 (dd, J=12 Hz, J=3 Hz, 1H), 3.76(t, J=6 Hz, 2H), 3.97(dd, J=12 Hz, J=3 Hz, 1H), 4.55(bs, 1H), 4.69(bs, 1H), 4.86(bs, 1.8H), 5.98(d, J=3 Hz, 1H), 8.19(s, 1H), 8.60(s, 1H). ¹³C-NMR (300 MHz, EtOD): δ 35.73, 36.00, 63.91, 65.64, 78.14, 84.35, 94.32, 142.87, 155.33, 156.68, 187.82. FAB-HRMS calcd for C₁₂H₁₇N₅O₄S (MH+) 364.1054, found, 364.1055.

3a Thiophenol; 3b 6-N-(phenylthiol)methyl-Cordycepin, Cordy 110 (210 mg, 57.2%).

¹H-NMR (300 MHz, DMSO): δ 1.91(m, 1H), 2.26(m, 1H), 3.49(d, J=6 Hz, 1H), 3.71(d, J=6 Hz, 1H), 4.35(bs, 1H), 4.55(bs, 1H), 5.05(bs, 1.6H?), 5.90(s, 1H), 7.22(m, 1H), 7.30(t, J=6 Hz, 2H), 7.44(d, J=7.2 Hz, 2H), 8.28(s, 1H), 8.32(s, 1H). FAB-HRMS calcd for C₁₇H₁₉N₅O₃S (MH+) 374.1276, found 374.1287.

4a p-Thiocresol; 4b 6-N-(4-methylbenzylthiol)methyl-Cordycepin, Cordy-106 (238.5 mg, 61.5%).

¹H-NMR (300 MHz, EtOD): δ 2.03 (m, 1H), 2.26(s, 3H), 2.38(m, 1H), 3.64(dd, J=12 Hz, J=3 Hz, 1H), 3.94(dd, J=12 Hz, J=3 Hz, 1H), 4.54(m, 1H), 4.65(m, 1H), 5.06(bs, 1.7H), 5.96(d, J=3 Hz 1H), 7.02(d, J=6 Hz, 2H), 7.34(d, J=6 Hz, 2H), 8.26(s, 1H), 8.45(s, 1H). ¹³C-NMR (300 MHz, EtOD): 21.08, 33.92, 46.90, 63.43, 76.29, 82.25, 93.34, 121.14, 130.27, 133.37, 137.99, 140.62, 152.90, 154.65. FAB-HRMS calcd for C₁₈H₂₁N₅O₃S (MH+) 388.1448, found 388.1443.

5a 4-methoxybenzenethiol; 5b 6-N-(4-methoxybenzothio)methyl-Cordycepin, Cordy-113(134.1 mg, 47.5%).

¹H-NMP (300 MHz, DMSO): δ 1.92(m, 1H), 2.25(m, 1H), 3.49 (m, 1H), 3.68(m, 1H), 3.73(s, 3H), 4.37 (m, 1H), 4.57 (bs, 1H), 4.95(bs, 1.5H), 5.15(bs, 0.78H), 5.71(d, 0.7H), 5.91 (d, J=7 Hz, 1H), 6.88(d, J=7.5 Hz, 2H), 7.40 (d, J=7.5 Hz, 2H), 8.31(s, 1H), 8.49(s, 1H), 8.65(m, 0.7H). FAB-HRMS calcd for C₁₈H₂₂N₅O₄S (MH+) 404.1403, found 404.1393.

6a 2-mercaptobenzoic acid; 6b 6-N-(1-para-benzoatethio)methyl-Cordycepin; Cordy-114

(138.1 mg mg, 44.8%).

¹H-NMR (300 MHz, DMSO): δ 1.91(m, 1H), 2.23(m, 1H), 3.54(d, J=15 Hz, 1H), 3.68 (d, J=15 Hz, 1H), 4.36 (bs, 1H), 4.56(bs, 1H), 5.08(bs, 2H), 5.71(bs, 0.56H), 5.91(s, 1H), 7.28(t, J=7.5 Hz, 1H), 7.51(t, J=7.5 Hz, 1H), 7.78(d, J=7.5 Hz, 1H), 7.84, (d, J=7.5 Hz, 1H), 8.36(s, 1H), 8.45(s, 1H), 8.87(bs, 0.7H), 13.25(bs, 0.85H). FAB-HRMS calcd for C₁₈H₂₀N₅O₅S (MH+) 418.1195, found 418.1185.

7a 4-nitrobenzenethiol; 7b 6-N-(4-nitrobenzylthio)methyl-Cordycepin; Cordy-115 (171.6 mg, 51.3%).

¹HNMR (300 MHz, DMSO): δ 1.92(m, 1H), 2.23(m, 1H), 3.53(m, 1H), 3.68(m, 1H), 4.35(s, 1H), 4.56(s, 1H), 5.12(bs, 1H), 5.24(bs, 1.6H), 5.90(s, 1H), 6.18(s, 1H), 7.73(d, J=7.5 Hz, 2H), 8.13 (d, J=8 Hz, 2H), 8.37(s, 1H), 8.45(s, 1H), 8.94(bs, 0.5H). FAB-HRMS calcd for C₁₇H₁₉N₆O₃S (MH+) 419.1134, found 419.1138.

8a 4-acetamidothiophenol; 8b 6-N-(4-N-acetylbenzylthio)methyl-Cordycepin; Cordy 116)

(145.1 mg, 49.6%). ¹HNMR (300 MHz, DMSO): δ 1.92(m, 1H), 2.02(s, 3H), 2.25(m, 1H), 3.53 (d, J=9 Hz, 1H), 3.67(m, 1H), 4.36 (bs, 1H), 4.57(bs, 1H), 4.98(bs, 1H), 5.13(bs, 0.8H), 5.70(d, J=2 Hz, 0.6H), 5.90(s, 1H), 7.38(d, J=6 Hz, 2H), 7.51(d, J=6 Hz, 2H), 8.31(d, J=1 Hz, 1H), 8.41(bs, 1H), 8.68(bs, 0.9H), 9.98(s, 1H). FAB-HRMS calcd for C₁₉H₂₃N₆O₄S (MH+) 431.1485, found 431.1502.

### EXAMPLE 2

### Enhanced Stability of Thioaminal Prodrugs

To analyze the half-life of thioaminal prodrugs, HPLC stability analysis was performed on the following Cordycepin prodrugs. Solutions (0.63 mM) of each of the following compounds was prepared in 30 mM phosphate buffer (pH 7.3) containing 5% DMSO and incubated at 37 C. The time point at which half of the Cordycepin prodrug was hydrolysed to form the parent nucleoside analog, Cordycepin (t ½) is listed in Table 1 below.

TABLE 1

| Cordycepin Prodrug | t½ (hours) |
|---|---|
| 1b | >72 |
| 2b | >72 |
| 3b | 15 |
| 5b | 17 |
| 7b | 1 |
| 8b | 28 |

### EXAMPLE 3

### Biological Activity of Thioaminal Prodrugs

The in vitro cytotoxicities of thioaminal prodrugs of Cordycepin were compared to the parent drug Cordycepin using a standard MTT assay of cell viability (Hansen et al. (1989) J. Immunol. Meth. 119:203-10). Leukemia cell lines growing in the exponential phase (MOLT, HL-69, and CEM/CI) were plated at a final concentration of 1×10⁵ cells/ml and exposed to serial dilutions of the Cordycepin prodrugs. In a subset of the experiments (denoted by “+”), the cells were preincubated with 1 μM of the ADA inhibitor 2′-deoxycoformycin for 30 minutes prior to addition of prodrug. Cell viability was determined after incubation for 5 days at 37 C by addition of 3-(4,5-dimethylthizaol-2-yl)-2,5-diplenyl-tetrazolium dye. IC50 was defined as the drug concentration that reduced cell viability 50% in comparison with the appropriate control. As Table 2 below illustrates, the Cordycepin prodrugs 3b, 4b, 5b and 8b were more potent cytotoxic agents than Cordycepin, both in the presence and absence of ADA inhibitor. Furthermore, the compound 3b was not significantly attenuated in the absence of ADA inhibitor, suggesting that it does not require protection from ADA in order to retain its biological activity.

TABLE 2

| | | |
|---|---|---|
| MOLT | HL-60 | CEM/Cl |
| IC50 (uM) | IC50 (uM) | IC50 (uM) |

| | − | + | − | + | − | + |
|---|---|---|---|---|---|---|
| Cordycepin | 80 | 21 | 42 | 9 | 27 | 2 |
| 3b | 18 | 16 | 24 | 7 | 5.5 | 2 |
| 4b | 35 | 30 | 50 | 17 | 24 | 5 |
| 5b | N/A | N/A | N/A | N/A | 15 | 3 |
| 8b | N/A | N/A | N/A | N/A | 15 | 3 |

The contents of all references, pending patent applications and published patents, cited throughout this application are hereby expressly incorporated by reference.
Equivalents

Those skilled in the art will recognize, or be able to ascertain using no more than routine experimentation, many equivalents to the specific embodiments of the invention described herein. Such equivalents are intended to be encompassed by the following claims.

1. A compound having the structure
a) A-Xₙ
wherein A is selected from the group consisting of a nucleotide, a nucleotide analog, a nucleoside, and a nucleoside analog, and wherein A comprises at least one amine, wherein the amine is derivatized with at least one X;
wherein n is either 1 or 2; and
wherein each X is independently selected from the group consisting of an aminal having the structure —(CR₄R₅)—O—R₆, a thioaminal having the structure —(CR₁R₂)—S—R₃, and combinations thereof, wherein each R is the same or different and is independently selected from the group consisting of a hydrogen, an alkyl, a substituted alkyl, an alkoxy, a substituted alkoxy, an aryl, and a substituted aryl; or 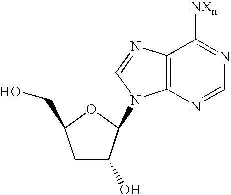
wherein n is either 1 or 2; and
wherein each X is independently selected from the group consisting of a hydrogen, an aminal having the structure —(CR₄R₅)—O—R₆, a thioaminal having the structure —(CR₁R₂)—S—R₃ , and combinations thereof, wherein at least one X is the thioaminal or the aminal, wherein each R is the same or different and is independently selected from the group consisting of a hydrogen, an alkyl, a substituted alkyl, an alkoxy, a substituted alkoxy, an aryl, and a substituted aryl.

2. The compound of claim 1, wherein A comprises a nucleoside analog.

3. The compound of claim 1, wherein A comprises cordycepin.

4. The compound of claim 1, wherein A is selected from the group consisting of adenosine, cytosine, fluoroarabinofluorcytosine, ganciclovir, trimethoprim, penciclovir, valaciclovir, vidarabine, arabinofuranosyladenine (Ara-A), arabinocytidine, acyclovir, arabinofuranosylcytosine (Cytarabine, Ara-C), arabinofuranosyl-5-fluorocytosine, cytidine, 2′-deoxycytidine, famciclovir, flucytosine, 5-fluorocytosine, 5′-fluoro-1′,2′-dioxalane cytosine (B-D-FDOC), 5-fluoro-2′3′-dideoxycytidine (D-D-FddC), 5-fluoro-2′,3′-dideoxy-2′,3′-didehydrocytosine (B-D-Fd4C), and 5-fluoro-3′deoxy-3′thiacytidine (B-D-FTC).

5. The compound of claim 1, wherein A is selected from the group consising of a nucleotide, a monophosphate nucleotide, a diphosphate nucleotide, a triphosphate nucleotide and analogs thereof.

6. The compound of claim 1, wherein the amine is a primary or secondary amine.

7. The compound of claim 1, wherein the amine is an aromatic amine.

8. (canceled)

9. The compound of claim 1, wherein X is capable of being hydrolyzed when the compound is administered to a subject.

10. The compound of claim 1, further comprising a targeting agent.

11. The compound of claim 10, wherein the targeting agent is selected from the group consisting of an antibody, an antibody fragment, a hormone, an aptamer and a peptide.

12. The compound of claim 1, wherein the compound comprises a solubility agent to modulate the solubility of the compound.

13. The compound of claim 12, wherein the solubility agent is selected from the group consisting of polyethylene glycol (PEG), phophate esters, phosphoramide esters, amino acid esters, t-BOC amino acids, lipids, steroids, amine-containing carbon chains, amino acids, and peptides.

14. The compound of claim 1, wherein n is 2, and wherein each X comprises a thioaminal.

15. The compound of claim 1, wherein n is 2, wherein one X comprises an aminal and wherein the other X comprises a thioaminal.

16. A composition comprising the compound of claim 1 and a carrier.

17. The pharmaceutical composition of claim 16, further comprising a second therapeutic agent.

18. The compound of claim 1, wherein the compound comprises the following structure:

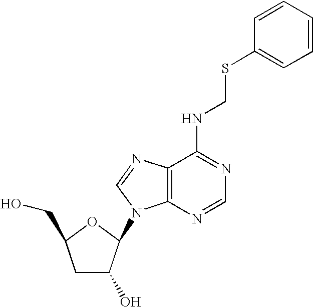

19. The compound of claim 1, wherein the compound comprises the following structure:

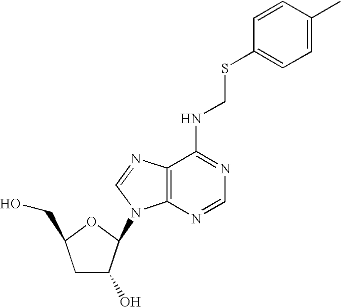

20. The compound of claim 1, wherein the compound comprises the following structure:

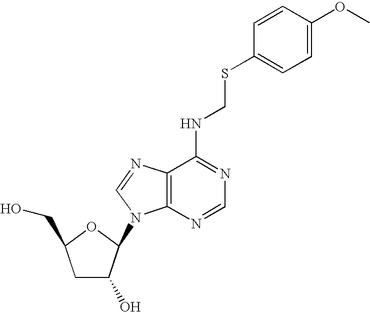

21. A method for protecting a compound comprising at least one free amine, the method comprising derivatizing the amine with at least one substituent, wherein the substituent is selected from the group consisting of an aminal having the structure —(CR₄R₅)—O—R₆, a thioaminal having the structure —(CR₁R₂)—S—R₃, and combinations thereof, wherein each R is the same or different and is independently selected from the group consisting of a hydrogen, an alkyl, a substituted alkyl, an alkoxy, a substituted alkoxy, an aryl, and a substituted aryl.

22. The method of claim 21, wherein the method protects the compound from deactivation.

23. The method of claim 22, wherein the method protects the compound from deactivation by a deaminase.

24. The method of claim 21, wherein the amine is a primary or secondary amine.

25. The method of claim 21, wherein the amine is an aromatic amine.

26. A method for treating a subject or a biological product with a neoplastic disorder, a parasite, or a fungal or fungal-like organism comprising administering to the subject or the biological product a therapeutically effective amount of the compound of claim 1.

27. The method of claim 26, wherein the neoplastic disorder is selected from the group consisting of a leukemia, lymphoma, sarcoma, carcinoma, neural cell tumor, squamous cell carcinoma, germ cell tumor, undifferentiated tumor, seminoma, melanoma, neuroblastoma, mixed cell tumor, metastatic neoplasia, terminal deoxynucleotidyl transferase-positive leukemia, or terminal deoxynucleotidyl transferase-positive lymphoma, and neoplasia due to pathogenic infections and malignancy.

28. (canceled)

29. The method of claim 26, wherein the parasite is selected from the group consisting of trypanasonal parasite, trypanasonal brucei, trypanasonal cruzi, plasmodium parasite, plasmodium falciparum, plasmodium vivax, plasmodium ovale and plasmodium malarie.

30. The method of claim 26, wherein the subject has a parasitemia which is undetectable after treatment.

31. (canceled)

32. The method of claim 26, wherein the subject has an organism load which is reduced at least 100-fold after treatment.

33. The method of claim 26, wherein the subject has a mycosis which is undetectable after treatment.

34. The method of claim 26, wherein the fungal organism is a eukaryotic organism.

35. The method of claim 26, wherein the organism is selected from the group consisting of Candida krusei, C. glabrata, C. albicans and C. tropicalis, C. parapsilosis, Trichophyton rubrum, T. mentagrophytes, T. tonsurans, Microsporum audouini, M. canis and T. floccosum, Nocardia asteroides and N. brasiliensis, Actinomyces israelii, species of the genera Mucor, Absidia, Rhizopus, Cunninghamella, Zygomycetes, Aspergillus fumigatus, A. flavus, A. niger, species of the genera Aspergillus, Crypttococcus neoformans, Paracoccidioides brasiliensis, Coccidioides immitis, Blastomycetes dermatitis and Histoplasma capsulatum.

36. The method of claim 26, wherein the fungal-like organism is a species of the genera Actinomyces or Nocardia.

37. The method of claim 26, wherein each X is capable of being hydrolyzed when the compound is administered to a subject.

38. The method of claim 26, wherein the compound further comprises a targeting agent.

39. The method of claim 38, wherein the targeting agent is selected from the group consisting of an antibody, an antibody fragment, a hormone, an aptamer, and a peptide.

40. The method of claim 26, wherein the compound comprises a solubility agent to modulate the solubility of the compound.

41. The compound of claim 40, wherein the solubility agent is selected from the group consisting of polyethylene glycol (PEG), phophate esters, phosphoramide esters, amino acid esters, t-BOC amino acids, lipids, steroids, amine-containing carbon chains, amino acids and peptides.

42. The method of claim 26, wherein A comprises a nucleoside analog.

43. The method of claim 26, wherein A comprises cordycepin.

44. The method of claim 26, wherein the compound is administered parenterally, sublingually, subcutaneously, intramuscularly, intrathecally, intraperitoneally, intrapleurally, enterally, by pulmonary absorption or by topical application.

45. The method of claim 44, wherein the parenteral administration is by intravenous injection.

46. The method of claim 26, wherein the compound is administered daily for multiple days.

47. The method of claim 26, wherein the therapeutically effective amount of the compound is between about 1 ng/kg subject weight to about 50 mg/kg subject weight.

48. The method of claim 26, wherein treatment results in a compound concentration in at least one measurable fluid of the subject of between about 0.1 nM to about 50 μM.

49. The method of claim 26, wherein the amine is a primary or secondary amine.

50. The method of claim 26, wherein the amine is an aromatic amine.

51. The method of claim 26, wherein the biological product comprises living cells.

52. The method of claim 51, wherein the living cells are selected from the group consisting of whole blood, fractionated blood, plasma, sentra, bone marrow and transplantable organs.

53. The method of claim 26, wherein the biological product is a food product.

54. The method of claim 53, wherein the food product is selected from the group consisting of rice, wheat, barley, corn, soybeans, breads, oils, sugars, spices, dairy products, alimentary paste, vegetables and fruit.

55. The method of claim 26, wherein the biological product is derived from living cells.

56. The method of claim 55, wherein the biological product derived from living cells is selected from the group consisting of cytokines, antibodies, immune system regulators, recombinant proteins and blood products.

57. The method of claim 26, wherein the biological product is incubated in a solution comprising the compound.

58. The method of claim 26, wherein the biological product is powdered with a powder comprising the compound.

59. The method of claim 26, wherein the subject is a mammal.

60. The method of claim 59, wherein the mammal is selected from the group consisting of humans, goats, camels, dogs, cats, cattle, monkeys, horses, pigs, and rodents.

61. The method of claim 26, further comprising co-administering the compound with a second therapeutic agent.

62. A method for protecting a free amine, comprising derivatizing the amine with at least one substituent selected from the group consisting of an aminal, a thioaminal, or a combination thereof.

63. The method of claim 62, wherein the thioaminal comprises the structure —(CR₁R₂)—S—R₃, wherein each R is the same or different and is independently selected from the group consisting of a hydrogen, an alkyl, a substituted alkyl, an alkoxy, a substituted alkoxy, an aryl, and a substituted aryl.

64. The method of claim 62, wherein the aminal comprises the structure —(CR₄R₅)—O—R₆, wherein each R is the same or different and is independently selected from the group consisting of a hydrogen, an alkyl, a substituted alkyl, an alkoxy, a substituted alkoxy, an aryl, and a substituted aryl.

65. A method for protecting a nucleoside, a nucleotide or an analog thereof, wherein the nucleoside, nucleotide or analog comprises at least one amine, comprising derivatizing the amine with at least one substituent selected from the group consisting of an aminal, a thioaminal, or a combination thereof.

66. The method of claim 65, wherein the thioaminal comprises the structure —(CR₁R₂)—S—R₃, wherein each R is the same or different and is independently selected from the group consisting of a hydrogen, an alkyl, a substituted alkyl, an alkoxy, a substituted alkoxy, an aryl, and a substituted aryl.

67. The method of claim 65, wherein the aminal comprises the structure —(CR₄R₅)—O—R₆, wherein each R is the same or different and is independently selected from the group consisting of a hydrogen, an alkyl, a substituted alkyl, an alkoxy, a substituted alkoxy, an aryl, and a substituted aryl.

68. A nucleotide, nucleoside or analog thereof, wherein the nucleotide, nucleoside or analog comprises at least one amine, and wherein the amine is derivatized with at least one substituent selected from the group consisting of an aminal, a thioaminal, or a combination thereof.

69. The nucleotide, nucleoside or analog of claim 68, wherein the thioaminal comprises the structure —(CR₁R₂)—S—R₃, wherein each R is the same or different and is independently selected from the group consisting of a hydrogen, an alkyl, a substituted alkyl, an alkoxy, a substituted alkoxy, an aryl, and a substituted aryl.

70. The nucleotide, nucleoside or analog of claim 68, wherein the aminal comprises the structure —(CR₄R₅)—O—R₆, wherein each R is the same or different and is independently selected from the group consisting of a hydrogen, an alkyl, a substituted alkyl, an alkoxy, a substituted alkoxy, an aryl, and a substituted aryl.
